(19) United States
(12) Patent Application Publication
(10) Pub. No.: US 2010/0272697 A1
(43) Pub. Date: Oct. 28, 2010

(54) PANCREATIC ISLET CELLS COMPOSITION AND METHODS

(76) Inventors: Ali Naji, North Wales, PA (US); Ming Yu, Wallingford, PA (US); Chengyang Liu, Philidelphia, PA (US); Hoorman Noorchashm, Narberth, PA (US); Omaida C. Velazquez, Miami, FL (US)

Correspondence Address: Pearl Cohen Zedek Latzer, LLP, 1500 Broadway, 12th Floor, New York, NY, 10036, US

(21) Appl. No.: 12/595,411

(22) PCT Filed: Apr. 10, 2008
(86) PCT No.: PCT/US08/04596

§ 371 Date: Jun. 15, 2010

Related U.S. Application Data
(60) Provisional application No. 60/907,583, filed on Apr. 10, 2007.

(51) Int. Cl. A61K 35/39; A61P 3/10

(52) U.S. Cl. 424/93.21; 424/93.7

(57) ABSTRACT

A composition comprising islet cells and an extracellular matrix component as well as methods of transplanting same are described. Retroperitoneal and subcutaneous islet cells transplants maintain their viability and increased insulin production for prolonged periods of time.

### FIELD OF THE INVENTION

The invention relates to composition comprising pancreatic islet cells and an extracellular matrix component and methods of transplanting same. Specifically, the invention is directed to compositions and methods for pancreatic islet cells transplantation, maintaining their viability and increasing insulin production for prolonged periods of time.

### BACKGROUND OF THE INVENTION

Diabetes mellitus is a medical disorder characterized by varying or persistent hyperglycemia (elevated blood sugar levels); Hypoglycemia (low blood sugar) is rarely a feature (except as an accident of treatment (usually misapplication of medication in particular circumstances)). While there are different types of diabetes mellitus, most are asymptomatic for a time after onset, but all share similar symptomatology and complications at advanced stages. This disease involves multiple causal factors and clinical aspects, all of which should be well understood for better management. Patient understanding and participation is highly desired as blood glucose levels change continuously in response to diet, exercise, physical and psychological stress, infection, accident (i.e., trauma), hormonal changes; Management of diabetes requires intensive lifestyle changes by the patient in order to achieve near-normal blood glucose, and the prevention of long term complications of the disease.

Hyperglycemia can lead to dehydration and ketoacidosis. Longer-term complications include cardiovascular disease (doubled risk—equal rates to those with heart attacks from advanced atherosclerotic disease), renal failure (worldwide, diabetes mellitus is the most common cause of chronic renal failure requiring renal dialysis), retinal damage with eventual blindness, nerve damage and eventual gangrene with probable loss of toes, feet, and even legs.

Conversely, successfully keeping blood sugar normal at all times, especially 0.5 to about 4 hours after eating, though difficult to do, has been compellingly shown to reduce/prevent each of these problems.

### SUMMARY OF THE INVENTION

This invention relates, in one embodiment, to a composition comprising islet cells and an extracellular matrix component.

In another embodiment, this invention provides a method for maintaining the viability of transplanted islet cells in-vivo, comprising the step of: transplanting a composition comprising islet cells and an extracellular matrix component into a subject.

In another embodiment, this invention provides a method for increasing insulin production in a subject, the method comprising: transplanting a composition comprising islet cells and an extracellular matrix component into said subject, thereby increasing insulin production in a subject.

In another embodiment, this invention provides a method of treating diabetes in a subject, the method comprising: transplanting a composition comprising islet cells and an extracellular matrix component into a subject, thereby treating diabetes in a subject.

In another embodiment, this invention provides a method of transplanting cells subcutaneously comprising the step of transplanting subcutaneously a composition comprising islet cells into a subject.

In another embodiment, this invention provides a method of transplanting cells retroperitoneally, comprising the step of transplanting a composition comprising islet cells into the retroperitoneal space of a subject.

### BRIEF DESCRIPTION OF THE FIGURES

FIG. 1. Shows the Survival period of islet isografts within a collagen matrix following subcutaneous transplantation. Serial histological analysis of islet-bearing anterior abdominal wall skin excision at >200 days following transplantation (A). H&E, immunohistochemistry for insulin (upper panel) and collagen (middle panel), and Aldehyde fuschin staining (lower panel) of serial sections. Random a.m. blood glucose levels in a cohort of B6 mice transplanted subcutaneously with syngeneic isolated islet (B). The arrows indicate the timing of islet-bearing anterior abdominal wall excision, which in all cases precipitated diabetes recurrence.

FIG. 2. Subcutaneously transplanted human islet in STZ-induced diabetic B6/scid recipients. Histological assessment of subcutaneously transplanted islets in the anterior abdominal wall of representative recipient B6/scid mice (A). H&E and immunohistochemistry for insulin (gray)/glucagon (black) is shown. Excision of the islet bearing anterior abdominal wall was performed at >200 following transplantation. Results of a glucose tolerance test of a 4 B6/scid mice, recipients of subcutaneously transplanted human islets, as compared to a control B6 mouse (B). This assay was performed at 120 days following transplantation. Demonstrates the serum human c-peptide levels in B6/scid mice, recipients of subcutaneously transplanted human islets, as compared to a control B6 mice (i.e., C-1 and C-2) (C). This assay was performed at 120 days following transplantation.

FIG. 3. Biopsy of abdominal wall of cynomolgus monkeys after autologous islet transplantation into the subcutaneous space. Serial sections of the subcutaneous islet implantation site in two different cynomolgus monkey recipients of autologous islets at 49 days (A) and 72 days (B) following transplantation. Serial sections were stained using H&E and immunohistochemistry using insulin (black) and glucagon (gray).

For simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Islet Cells

In one embodiment, the present invention provides a composition comprising islet cells and an extracellular matrix (ECM) component (Example 6). In one embodiment, the present invention provides a composition comprising islet cells cultured in an extracellular matrix (ECM) component. In another embodiment, the present invention provides a composition comprising islet cells. In another embodiment, the present invention provides a composition comprising islet cells and a medium. In one embodiment, islet cells are obtained from irregularly shaped patches of endocrine tissue in the pancreas. In one embodiment, islet cells are derived from the pancreas. In one embodiment, islet cells are derived from a human pancreas. In one embodiment, the islet cells of the present invention comprise β cells. In one embodiment, β cells produce insulin thus regulating blood glucose. In some embodiments, islet cells are comprised of α cells which produce glucagon, β cells, Delta cells which produce somatostatin, and another type of cell which secretes a pancreatic polypeptide which slows down nutrient absorption.

In some embodiments, islet cells are selected from the group consisting of primary pancreatic islet cells, pancreatic islet cell lines, genetically-transformed islet cells, islet cells obtained from neoplastic sources, or fetal islet cells.

In one embodiment, primary islet cells are obtained from a pancreas as described in Example 4. In one embodiment, the primary pancreatic islet cells population comprises two sub-populations wherein one sub-population is enriched for β cells. In one embodiment, the terms “enriched for β cells” and “substantially β cells” are interchangeable. In one embodiment, a composition wherein islet cells are substantially β cells comprises at least 70% β cells. In another embodiment, a composition wherein islet cells are substantially β cells comprises at least 75% β cells. In another embodiment, a composition wherein islet cells are substantially β cells comprises at least 80% β cells. In another embodiment, a composition wherein islet cells are substantially β cells comprises at least 85% β cells. In another embodiment, a composition wherein islet cells are substantially β cells comprises at least 90% β cells. In another embodiment, a composition wherein islet cells are substantially β cells comprises at least 95% β cells. In another embodiment, a composition wherein islet cells are substantially β cells comprises at least 97% β cells.

In some embodiments, pancreatic islet cell lines are derived from rodents. In one embodiment, a pancreatic islet cell line is derived from mouse. In one embodiment, a pancreatic islet cell line is derived from rat. In some embodiments, pancreatic islet cell lines are derived from primates. In one embodiment, a pancreatic islet cell line is derived from chimpanzee. In one embodiment, a pancreatic islet cell line is derived from baboon. In some embodiments, pancreatic islet cell lines are derived from human.

In some embodiments, the islet cells of the present invention are genetically-modified islet cells. In some embodiments, genetically-modified islet cells are genetically-transformed islet cells. In some embodiments, genetically-transformed islet cells become malignant cells in cell culture. In one embodiment, the methods of transforming primary islet cells are well known to a person of average skill in the art.

In some embodiments, pancreatic β cells are produced from non-β pancreatic cells by providing for production of an islet transcription factor in a pancreatic cell either in vivo (e.g., by administration of islet transcription factor-encoding nucleic acid (e.g., RNA or DNA) to the pancreas of a subject, e.g., by introduction of nucleic acid into a lumen of a pancreatic duct), or in vitro, e.g., by contacting a target cell (e.g., an isolated, non-beta, pancreatic cell) with islet transcription factor-encoding nucleic acid (e.g., RNA or DNA) in culture (which cells are then cultured, expanded, and transplanted into a subject).

In one embodiment, β cells are produced by providing for expression of neurogenin3 (Ngn3) at a level sufficient to induce the beta cell phenotype in the target cell. Expression of Ngn3 in the target cell can be accomplished in a variety of ways. In one embodiment, Ngn3 expression is accomplished by introduction of Ngn3-encoding nucleic acid (e.g., DNA or RNA) to provide for expression of the encoded Ngn3 polypeptide in the target cell. In another embodiment, Ngn3 expression is induced by introduction of a gene encoding a protein that provides for induction of Ngn3 expression (e.g., expression of an “upstream” positive regulator of Ngn3 expression in the target cell). In another embodiment, Ngn3 expression is accomplished by introduction of a gene encoding a protein that inhibits activity (e.g., function or expression) of a negative regulator of Ngn3 expression. In another embodiment, Ngn3 expression is induced by introduction of a small molecule that provides for induction of Ngn3 expression (e.g., a small molecule pharmaceutical that induces Ngn3 expression in the target cell).

In one embodiment, the islet cells are obtained from neoplastic sources. In one embodiment, the neoplastic source is a malignant carcinoma that is located in the islet cells of the pancreas. In one embodiment, neoplasms of the endocrine pancreas are divided into functional and nonfunctional varieties. In one embodiment, the pancreatic endocrine neoplasm is functional, indicating that it elaborates one or more hormonal products into the blood, leading to a recognizable clinical syndrome.

### Fetal Islet Cells

In some embodiments, fetal islet cells are derived from rodents. In one embodiment, fetal islet cells are derived from mouse. In one embodiment, fetal islet cells are derived from rat. In some embodiments, fetal islet cells are derived from primates. In one embodiment, fetal islet cells are derived from chimpanzee. In one embodiment, fetal islet cells are derived from baboon. In one embodiment, fetal islet cells are derived from pig. In some embodiments, fetal islet cells are derived from human. In one embodiment, fetal islet cells are pancreatic progenitor cells.

In some embodiments, pancreatic progenitor cells of this invention are isolated from human fetal pancreatic tissue. In one embodiment, the age of the fetus is between about week 6 and about week 40. In another embodiment, the age of the fetus is between about week 8 and about week 26. In another embodiment, the age of the fetus is between about week 12 and about week 22.

In one embodiment, the pancreatic islet cells are differentiated from appropriate stem cells. A “stem cell” refers in one embodiment to a undifferentiated cell which is capable of essentially unlimited propagation either in vivo or ex vivo and capable of differentiation to other cell types. This can be in another embodiment to certain differentiated, or committed, immature, progenitor, or mature cell types present in the tissue from which it was isolated in other embodiments, or dramatically differentiated cell types, such as for example the islet cells that derive from a common precursor cell, or even to cell types at any stage in a tissue completely different from the tissue from which the stem cell is obtained. In one embodiment, blood stem cells may become brain cells or liver cells, neural stem cells can become blood cells, such that stem cells are pluripotential, and given the appropriate signals from their environment, they can differentiate into any tissue in the body. Accordingly and in one embodiment, the β-cells used in the compositions and methods described herein, are derived from pluripotent liver stem cells induced to differentiate.

In another embodiment, the pancreatic islet cells are differentiated from IPSCs, referring to Islet Producing Stem Cells. IPSCs are a small population of cells derived from ductal epithelium (i.e., pancreas-derived) discovered in fetal or adult pancreas which, in one embodiment, have the capacity of giving rise in vitro to IPSC undifferentiated progeny or to islet progenitor cells (IPCs), which in another embodiment give rise to islet-like structures or IPC-derived islets (IdIs). IPCs are pluripotent ion one embodiment, and committed to give rise to the differentiated cells of the in vivo islets of Langerhans and the ldis.

In some embodiments, fetal pancreatic tissue is microdissected. In one embodiment, non-limiting examples of microdissection include devices that render mechanical shearing forces (i.e. homogenizer, mortar and pestle, blender, etc.), devices that render cuts or tears (i.e. scalpel, syringes, forceps, etc.), or ultrasonic devices. In another embodiment, another method of microdissecting fetal pancreatic tissue is the use of enzyme treatment. In one embodiment, various enzyme treatments used to microdissect tissue are well known in the art. In one embodiment, one method includes the use of collagenase-dispase to digest partially sheared pancreatic tissue in a buffered medium that will sustain viability of cells isolated from the pancreatic tissue. In one embodiment, a concentration of at least about 0.5 mg/ml collagenase-dispase is used. In one embodiment, a concentration of at least about 1 mg/ml collagenase-dispase is used. In one embodiment, a concentration of at least about 5 mg/ml collagenase-dispase is used. In one embodiment, the amount of enzyme will depend on the age of the fetus and how large the pancreatic tissue is. In one embodiment, pancreatic tissue from fetus between about 14 weeks and about 22 weeks is digested with about 5 mg/ml of collagenase-dispase.

In one embodiment, the transplanted islet cells of the present invention comprise properties such as longevity and self-renewal. In some embodiments, the transplanted islet cells are able to produce insulin. In one embodiment, the transplanted islet cells are viable for at least a day after being transplanted. In another embodiment, the transplanted islet cells are viable for at least three days after being transplanted. In another embodiment, the transplanted islet cells are viable for at least seven days after being transplanted. In another embodiment, the transplanted islet cells are viable for at least fourteen days after being transplanted. In another embodiment, the transplanted islet cells are viable for at least thirty days after being transplanted. In another embodiment, the transplanted islet cells are viable for at least sixty days after being transplanted. In another embodiment, the transplanted islet cells are viable for at least one hundred and eighty days after being transplanted.

In some embodiments, pancreatic progenitor cells of the invention have the capacity to be passaged multiple times in a preferred serum-free nutrient media of the invention. In some embodiments, multipotency is retained during each passage and at any point after each passage, pancreatic progenitor cells of this invention can differentiate into functional exocrine or endocrine cells. In some embodiments, at any point after each passage, pancreatic progenitor cells may be used as an immunogen, for cell therapy, for bioassays, to establish a human pancreatic model, or for drug discovery and/or any method as disclosed herein.

In some embodiments, pancreatic progenitor cells of the invention can differentiate into exocrine or endocrine cells upon transplantation. Islet cells and/or pancreatic progenitor cells can be grown either in cell aggregates or in monolayers and then in some embodiments, combined with an ECM component.

In some embodiments, the same methods described for human pancreas can be utilized for pig or a primate pancreas as primate and pig pancreases share high similarity with human pancreases. In one embodiment, these methods are utilized to isolate viable islets of Langerhans from pig pancreases for experiment and transplantation into humans.

In one embodiment, the islet cells of the invention have the ability to secrete insulin in response to glucose. In one embodiment, islet cells secrete insulin in response to a medium, e.g., glucose-containing medium comprises about 4 mM, 6 mM, 8 mM, or 10 mM glucose.

In one embodiment, the composition of the present invention comprises at least 10⁴ islet cells. In one embodiment, the composition of the present invention comprises at least 10⁵ islet cells. In one embodiment, the composition of the present invention comprises at least 10⁶ islet cells.

### ECM

In some embodiments, the ECM component is a structural protein. In one embodiment, the structural protein is collagen. In another embodiment, the structural protein is elastin. In some embodiments, the ECM component is a specialized protein. In another embodiment, the specialized protein is fibrillin. In another embodiment, the specialized protein is fibronectin. In another embodiment, the specialized protein is laminin. In some embodiments, the ECM component is a proteoglycan. In one embodiment, proteoglycans are composed of a protein core to which is attached long chains of repeating disaccharide units termed glycosaminoglycans (GAGs) forming extremely complex high molecular weight components.

In one embodiment, collagen is collagen type I. In one embodiment, collagen type I comprises [a1(I)]₂[α(I)] chains. In one embodiment, collagen type I is derived from skin, tendon, or bone.

In one embodiment, collagen is collagen type II. In one embodiment, collagen type II comprises [α1(II)]₃ chains. In one embodiment, collagen type II is derived from cartilage or vitreous humor. In one embodiment, type II collagen fibrils are cross-linked to proteoglycans in the matrix by type IX collagen.

In one embodiment, collagen is collagen type III. In one embodiment, collagen type III comprises [α1(III)]₃ chains. In one embodiment, collagen type III is derived from skin or muscle, and is frequently found with type I collagen.

In one embodiment, collagen is collagen type IV. In one embodiment, collagen type IV comprises [α1(I)]₂[α2(IV)] chains. In one embodiment, collagen type IV is derived from basal lamina.

In one embodiment, collagen is collagen type V. In one embodiment, collagen type V comprises [α1(V)][α2(V)][α3(V)] chains. In one embodiment, collagen type V is derived from an interstitial tissue associated with type I collagen.

In one embodiment, collagen is collagen type VI. In one embodiment, collagen type VI comprises [α1(VI)][α2(VI)][α3(VI)] chains. In one embodiment, collagen type VI is derived from an interstitial tissue associated with type I collagen. In one embodiment, type VI collagen consists of relatively short triple-helical regions about 60 nm long separated by globular domains about 40 nm long. In some embodiments, fibrils of pure type VI collagen form a structure similar to beads on a string.

In one embodiment, collagen is collagen type VII. In one embodiment, collagen type VII comprises [α1(VII)]₃ chains. In one embodiment, collagen type VII is derived from epithelia.

In one embodiment, collagen is collagen type VIII. In one embodiment, collagen type VIII comprises [α1(VIII)]₃ chains. In one embodiment, collagen type VII is derived from endothelial cells.

In one embodiment, collagen is collagen type IX. In one embodiment, collagen type IX comprises [α1(IX)][α2(IX)][α3(IX)] chains. In one embodiment, collagen type IX is derived from cartilage, associated with type II collagen.

In one embodiment, collagen is collagen type X. In one embodiment, collagen type X comprises [α1(X)]₃ chains. In one embodiment, collagen type X is derived from hypertrophic and mineralizing cartilage.

In one embodiment, collagen is collagen type XI. In one embodiment, collagen type XI comprises [α1(XI)][α2(XI)][α3(XI)] chains. In one embodiment, collagen type XI is derived from cartilage.

In one embodiment, collagen is collagen type XII. In one embodiment, collagen type XII comprises [α1(XII)] chains. In one embodiment, collagen type XII is derived from sites wherein types I and III collagens are present.

In one embodiment, type I collagen molecules pack together side-by-side, forming fibrils with a diameter of 50-200 nm. In some embodiments, fibrils and adjacent collagen molecules are displaced from one another by 67 nm, about one-quarter of their length. In some embodiments, collagens types I, II, III, and V form rodlike triple helices due to side-by-side interactions.

In one embodiment, the collagen of the present invention is derived from cows. In another embodiment, collagen of the present invention is derived from patient's own fat or hyaluronic acid.

In one embodiment, collagen is a collagen-like substance which has been modified by dissolving collagen in water and modifying the thusly dissolved collagen to render its surface charge effectively more positive than prior to modification. In one embodiment, this material is well known and is disclosed, e.g., in U.S. Pat. No. 4,238,480. In another embodiment, modified collagen is freeze-dried to form a solid mass of gelatin. In some embodiments, the mass of gelatin may be formed in the shape of a rod, strip, film or flake.

In another embodiment, other forms of collagen which are suitable for use in the present invention include Semed F, a collagen preparation manufactured in native fiber form without any chemical or enzymatic modifications, and Semed S, a lyophilized collagen powder extracted from fresh bovine hides. In one embodiment, the Semed F material is a Type I collagen (greater than 95%), while the Semed S is a mixture of Type I and Type III collagen macro-molecules in which the shape and dimension of tropocollagen in its natural helical orientation is retained.

In some embodiments, the concentration of the collagen in the liquid which is to be freeze-dried can range from 0.5-10% and preferably 1-5%, with the lower concentrations forming less dense or discontinuous solids. In one embodiment, at lower concentrations of 0.5 to 1%, the Semed F forms a structure which approximates dense cobwebs.

In some embodiments, native collagen film, wherein the film strength is preserved and the triple-helix structure of the collagen polymer is maintained intact, can also be used, either alone or with a plasticizer incorporated therewith.

In some embodiments, gelatin or other water soluble forms of collagen are utilized. In one embodiment, soluble forms of collagen will readily polymerize at body temperatures to form a stable subcutaneous gel. In one embodiment, when soluble forms of collagen are implanted into the body, the polymerized material will become rapidly populated by host fibroblasts. In some embodiments, the material becomes vascularized and can remain histologically stable for at least 2 months. In another embodiment, the material becomes vascularized and can remain histologically stable for at least 4 months. In another embodiment, the material becomes vascularized and can remain histologically stable for at least 6 months. In another embodiment, the material becomes vascularized and can remain histologically stable for at least 8 months. In another embodiment, the material becomes vascularized and can remain histologically stable for at least 10 months. In another embodiment, the material becomes vascularized and can remain histologically stable for at least 12 months. In another embodiment, the material becomes vascularized and can remain histologically stable for at least 15 months. In another embodiment, the material becomes vascularized and can remain histologically stable for at least 18 months.

In some embodiments, the present invention provides mixtures of the various types of collagen to obtain the most desirable features of each grade.

In one embodiment, fibronectins are dimers of 2 similar peptides. In one embodiment, each chain of a fibronectins is 60-70nm long and 2-3nm thick. In another embodiment, fibronectins contain at least 6 tightly folded domains each with a high affinity for a different substrate such as heparan sulfate, collagen (separate domains for types I, II and III collagens), and fibrin and cell-surface receptors.

In one embodiment, laminin molecule is a heterotrimer assembled from α, β, and γ-chains. In some embodiments, laminins form independent networks and are associated with type IV collagen networks via entactin, and perlecan. In some embodiments, laminins contribute to cell viability, attachment, and differentiation, cell shape and movement, maintenance of tissue phenotype, and promotion of tissue survival.

In one embodiment, proteoglycans comprise chondroitin sulfate and dermatan sulfate chains. In another embodiment, proteoglycans comprise heparin and heparan sulfate chains. In another embodiment, proteoglycans comprise keratan sulfate chains. In one embodiment, proteoglycans are aggrecans, the major proteoglycan in cartilage. In another embodiment, proteoglycans are versican, present in many adult tissues including blood vessels and skin. In another embodiment, proteoglycans are small leucine rich repeat proteoglycans (SLRPs). In some embodiments SLRPs include decorin, biglycan, fibromodulin, and lumican.

In one embodiment, the composition of the present invention further comprises an appropriate composition, such as those described herein, wherein, islet cells can be induced to proliferate and generate islet cell progeny. In one embodiment, the term composition in which islet cells progeny are placed refers to the combination of external or extrinsic physical and/or chemical conditions that affect and influence the growth, development, and differentiation of islet cells. In one embodiment, the composition can be ex-vivo or in-vivo. In one embodiment, appropriate composition, such as those described herein, wherein, islet cells can be induced to differentiate into insulin secreting islet cell are included. In another embodiment, the composition is ex-vivo and comprises islet cells placed in cell culture medium in an incubator.

In some embodiments, wide variety of basal cell-sustaining medium can be used to keep the pH of the liquid in a range that promotes survival of pancreatic progenitor cells or islet cells. In one embodiment, the medium comprises F12/DMEM, Ham's F10, CMRL-1066, Minimal essential medium (MEM, Sigma), RPMI-1640 (Sigma), Dulbecco's Modified Eagle's Medium, and Iscove's Modified Eagle's Medium (IMEM). In some embodiments, any of the basal nutrient medium described in Ham and Wallace Meth. Enz., 58:44 (1979), Barnes and Sato Anal. Biochem., 102:255 (1980), or Mather, J. P. and Roberts, P. E. “Introduction to Cell and Tissue Culture”, Plenum Press, New York (1998) can also be used.

In some embodiments, the medium is further supplemented with insulin, transferrin, epidermal growth factor, ethanolamine, phosphoethanolamine, selenium, triiodothyronine, progesterone, hydrocortisone, forskolin, heregulin, aprotinin, bovine pituitary extract, and gentamycin.

In one embodiment, the following amounts of nutrients are used to promote pancreatic progenitor cell and/or islet cell survival and growth: at least about 1 μg/ml insulin and not more than about 100 μg/ml insulin; or about 10 μg/ml insulin; at least about 1 μg/ml transferrin and not more than about 100 μg/ml transferrin, or about 10 μg/ml transferrin; at least about 1 ng/ml epidermal growth factor and not more than about 100 ng/ml epidermal growth factor, or about 5 ng/ml epidermal growth factor; at least about 1×10⁻⁸ M ethanolamine and not more than about 1×10⁻² M ethanolamine, or about 1×10⁻⁶ M ethanolamine; at least about 1×10⁻⁹ M phosphoethanolamine and not more than about 1×10⁻¹ M phosphoethanolamine, or about 1×10⁻⁶ M phosphoethanolamine; at least about 1×10⁻¹² M selenium and not more than about 1×10⁻¹ M selenium, or about 2.5×10⁻⁸ M selenium; at least about 1×10⁻¹⁵ M triiodothyronine and not more than about 5×10⁻¹ M triiodothyronine, or about 1×10⁻¹² M triiodothyronine; at least about 1×10⁻¹³ M progesterone and not more than about 1×10⁻¹ M progesterone, or about 1×10⁻⁹ M progesterone; at least about 1×10⁻¹⁵ M hydrocortisone and not more than about 1×10⁻¹ M hydrocortisone, or about 1×10⁻⁹ M hydrocortisone; at least about 0.001 μM forskolin and not more than about 50 μM forskolin, or about 1 μM forskolin; at least about 0.1 nM heregulin and not more than about 100 nM heregulin, or about 10 nM heregulin,; at least about 1 μg/ml aprotinin and not more than about 100 μg/ml aprotinin, or about 25 μg/ml aprotinin; at least about 1 μg/ml bovine pituitary extract and not more than about 500 μg/ml bovine pituitary extract, or about 75 μg/ml bovine pituitary extract; at least about 1 μg/ml gentamycin and not more than about 1 mg/ml gentamycin, or about 100 μg/ml gentamycin.

In one embodiment, the composition comprises cell culture medium comprising DMEM. In another embodiment, the cell culture medium further comprises 5-50 mM glucose. In another embodiment, the cell culture medium further comprises 0.2-10% (vol/vol) penicillin streptomycin. In another embodiment, the cell culture medium further comprises methylcellulose in a final concentration of less than 5%, or less than 1.5%. In some embodiments, the medium is supplemented with 5-30% fetal bovine serum (FBS). In some embodiments, the medium is supplemented with 30-70% medium derived from cultures of fibroblasts. In some embodiments, islet cells are cultivated at 35° C.-40° C. in a humidified incubator in an atmosphere of 95% air 5% CO₂. In one embodiment, islet cells are cultivated at 37° C. in a humidified incubator in an atmosphere of 95% air 5% CO₂.

In one embodiment, the cell culture medium is further supplemented with entactin. In one embodiment, entactin further supports matrix assembly.

In one embodiment, the cell culture medium is further supplemented with polylysinearginine, polylysine, proline, nicotinamide, transferring, insulin, insulin-like growth factors, glucocorticoid steroid, L-glutamine, D-galactose, D-glucose, or a mixture thereof.

Another aspect of the present invention contemplates a method for stimulating or otherwise facilitating formation of colonies of islet cells containing insulin-secreting cells. In one embodiment, the medium for culturing islet cells further comprises laminin-1 or a laminin-1-containing ECM or a functional derivative, homologue, mimetic, analogue or agonist thereof. In one embodiment, laminin-1 induces, supports, or maintains colonies comprising insulin-secreting cells.

In one embodiment, the medium for culturing islet cells further comprises a Bone Morphogenic Protein (BMP) or a functional derivative or homologue, mimetic, analogue or agonist thereof. In one embodiment, BMP induces, supports, or maintains colonies comprising insulin-secreting cells.

In some embodiments, a “BMP” or a specific BMP such as but not limited to “BMP 2”, “BMP 3”, “BMP 4”, “BMP 5”, “BMP 6” and “BMP 7” includes reference to a polypeptide having BMP properties including the ability to stimulate or otherwise facilitate the formation of insulin-secreting cells. BMPs contemplated herein are those belonging to the TGF β family of molecules. In some embodiments, these terms also encompass functional derivatives, homologues, mimetics and analogues of the BMP molecule including homodimeric and heterodimeric forms. In another embodiment, a derivative of a BMP is a mutant, part, portion or fragment including a BMP carrying a single or multiple amino acid substitution, addition and/or deletion to its amino acid sequence. In some embodiments, derivatives, homologues, mimetics and analogues are considered functional in that they are capable of stimulating or otherwise facilitating formation of insulin-secreting cells. In one embodiment, a derivative may also include an agonist or antagonist.

In one embodiment, the present invention provides that a BMP or combination of BMPs and/or laminin-1 or laminin-1-containing ECM alone or laminin-1 or laminin-1-containing ECM and a BMP are present in a composition of islet cells to facilitate the formation of colonies containing insulin-producing β cells. In another embodiment, the concentration of the BMPs present in a culture of islet cells may be modulated such that the cell culture is exposed to a regimen of BMPs and laminin-1 or laminin-1-containing ECM to stimulate the production of insulin-producing cells. In one embodiment, the BMPs may be used either individually or together with each other or with any other member of the BMP family for the purpose stimulating the formation of insulin-producing cells.

In one embodiment, the composition further comprises immunosuppresents selected from: calcineurin inhibitors, rapamycin, dacliximab (Zenapax), sirolimus (Rapamune), tacrolimus (Prograf), or a combination thereof.

In one embodiment, the composition further comprises Exendin-4 which is a homolog of GLP-1. In one embodiment, Exendin-4 increases β cell replication and differentiation. In one embodiment, the composition further comprises betacellulin. In some embodiments, Exendin-4 and betacellulin induce insulin transcription.

In one embodiment, the composition further comprises laminin. In one embodiment, a laminin 5 rich matrix induces higher insulin secretion in response to glucose.

In one embodiment, TGF α EGF and/or agents which interfere with TGF β activity, e.g., TGF β binding, are used to promote proliferation of adult or differentiated islet cells.

In one embodiment, the composition according to this aspect of the present invention may be referred to as a “pharmaceutical composition”. In another embodiment, the preparation of pharmaceutical compositions is well known in the art and reference can conveniently be made to Remington's Pharmaceutical Sciences, Mack Publishing, Company, Easton, Pa., USA.

In some embodiments, the composition further comprises pharmaceutically acceptable carriers and/or diluents including any and all solvents, dispersion medium, coatings, antibacterial and antifungal agents, isotonic and absorption delaying agents and the like. In one embodiment, the use of such medium and agents for pharmaceutical active substances is well known in the art.

In another embodiment, the present invention provides a two part pharmaceutical pack comprising a first compartment comprising the composition of the present invention and islet cells and a second compartment comprising an ECM component. In one embodiment, the contents of both compartments are mixed together prior to use or are prepared separately and administered simultaneously.

### Administration

In one embodiment, administration may be by any number of means including intravenous, intraperitonealy, retroperitoneal, intramuscular, or subcutaneous. In some embodiments, the composition is in a liquid form. In some embodiments, the composition is in a solid form. In some embodiments, administration is via a pump or injection. In one embodiment, delivery is “on-site” such as during surgery, biopsy or other interventionist therapy. In another embodiment, targeted delivery may also be accomplished.

In one embodiment, the islet cells are labeled. In one embodiment, the islet cells are labeled prior to transplantation. In one embodiment, islet cells of the invention are labeled by transfection with a fluorescent protein. In some embodiments, the identifiable gene product comprises various fluorescent proteins as will be known to one of skill in the art. In one embodiment, the identifiable gene product comprises a luminescent protein. In one embodiment, the luminescent protein is luciferase. In one embodiment, isotopes are used for tracking the transplanted islet cells in an animal model. In some embodiments, the isotopes comprise ³²P, ¹²⁵I, ¹²⁴I, ¹⁴C, ¹⁰⁹Cd, ⁵¹Cr, ⁶⁷Cu, ¹⁷⁹Ta, ¹¹¹In, ¹⁸F, or combinations thereof. In one embodiment a magnetic label is used for cell detection.

### Treatment

In one embodiment, the methods of present invention apply to patients suffering from diabetes. In one embodiment, the methods of present invention apply to patients suffering from diabetes type 1. In one embodiment, the methods of present invention apply to patients that are genetically at risk from developing type 1 diabetes or a related condition or who are at risk for non-genetic reasons such as age. In one embodiment, the methods of present invention extend to the treatment and/or prophylaxis of type 1 diabetes or a related condition.

In one embodiment, the methods of present invention provide that the composition of the present invention is transplanted to patient in need thereof. In one embodiment, the methods of present invention provide that during transplantation, a radiologist uses ultrasound and radiography to guide placement of a catheter to the transplantation site. In one embodiment, the methods of present invention provide that the composition of the present invention is infused through the catheter into the transplantation site. In one embodiment, the methods of present invention provide that the patient receives a local anesthetic. In some embodiments, the methods of present invention provide that the composition of the present invention is introduced through a small incision.

In one embodiment, the methods of present invention provide allotransplantation of islet cells. In one embodiment, the methods of the present invention provide that allotransplantation of islet cells comprises locating a genetically compatible donor. In one embodiment, the methods of present invention provide a xenotransplantation of islet cells. In one embodiment, the methods of present invention provide that xenotransplantation of islet cells comprise a rodent donor. In some embodiments, the methods of present invention provide that the rodent donor is selected from a group comprising rat, mouse, or a guinea pig. In one embodiment, the methods of present invention provide that xenotransplantation of islet cells comprise a pig donor. In one embodiment, the methods of present invention provide that xenotransplantation of islet cells comprise a bovine donor. In some embodiments, the methods of present invention provide that hyperacute rejection associated with xenotransplantation is solved by the use of transgenic animals.

### Methods for Maintaining the Viability of Cells

In one embodiment the present invention provides a method for maintaining the viability of transplanted islet cells in-vivo. In one embodiment, the methods of the present invention provide that the association of islet cells with an ECM component promotes the viability of the islet cells of the invention. In one embodiment, the methods of present invention provide that the association of islet cells with an ECM component promotes the viability of islet cells ex-vivo compared to islet cells grown in the absence of an ECM component. In one embodiment, the methods of present invention provide that the association of islet cells with an ECM component promotes the viability of islet cells de-novo after transplantation, compared to islet cells transplanted in the absence of an ECM component.

In one embodiment, the methods of present invention provide that transplanting a composition of the present invention subcutaneously promotes islet cells viability, compared to other modes of transplantation. In one embodiment, the methods of present invention provide that transplanting a composition of the present invention retroperitoneally promotes islet cells viability, compared to other modes of transplantation.

### Insulin Secretion

In one embodiment, the methods of present invention provide that the association of islet cells with an ECM component further promotes islet cells insulin secretion. In one embodiment, the methods of present invention provide that islet cells express increased levels of insulin as compared to dedifferentiated cells. In another embodiment, the methods of present invention provide that islet cells express increased levels of insulin as compared to islet cells growing in the absence of an ECM component. In one embodiment, the methods of present invention provide that the association of islet cells with an ECM component further promotes islet cells insulin secretion ex-vivo. In one embodiment, the methods of present invention provide that the association of islet cells with an ECM component further promotes islet cells insulin secretion de-novo, after transplantation. In one embodiment, the methods of present invention provide that transplanting a composition of the present invention subcutaneously promotes islet cells insulin secretion compared to other modes of transplantation. In one embodiment, the methods of present invention provide that transplanting a composition of the present invention retroperitoneally promotes islet cells insulin secretion compared to other modes of transplantation.

### Treating Diabetes

In one embodiment, the methods of present invention provide that the association of islet cells with an ECM component promotes the viability of islet cells de-novo after transplantation, thereby treating diabetes. In one embodiment, the methods of present invention provide that subcutaneous transplantation of a composition of the present invention promotes islet cells viability thereby treating diabetes. In one embodiment, the methods of present invention provide that transplanting a composition of the present invention retroperitoneally promotes islet cells viability thereby treating diabetes.

In one embodiment, the methods of present invention provide that the association of islet cells with an ECM component further promotes insulin secretion of the islet cells of the present invention, thereby treating diabetes. In one embodiment, the methods of present invention provide that the association of islet cells with an ECM component further promotes islet cells insulin secretion de-novo, after transplantation, thereby treating diabetes.

### Anatomic Sites for Isolated Islet Transplantation

In one embodiment, the methods of present invention provide that the anatomic implantation site is the peritoneal cavity. In another embodiment, the methods of present invention provide that the anatomic implantation site is the liver. In another embodiment, the methods of present invention provide that the anatomic implantation site is the renal subcapsule. In another embodiment, the methods of present invention provide that the anatomic implantation site is the epididymal fat pad. In another embodiment, the methods of present invention provide that the anatomic implantation site is the thymus. In another embodiment, the methods of present invention provide that the anatomic implantation site is the spleen. In another embodiment, the methods of present invention provide that the anatomic implantation site is the omental pouch. In another embodiment, the methods of present invention provide that the anatomic implantation site is a muscle. In another embodiment, the methods of present invention provide that the anatomic implantation site is the testes. In another embodiment, the methods of present invention provide that the anatomic implantation site is the portal venous circulation.

In another embodiment, the islet allograft is introduced via a percutaneous transhepatic approach, or via a mini-laparotomy. In another embodiment, the methods of present invention utlizing this anatomic implantation site may involve the adverse events of: 1) Procedure related complications and 2) the deleterious effect of the intra-hepatic milieu on islet engraftment and long-term function. In another embodiment, the procedure related morbidities include intra-peritoneal hemorrhage and portal vein thrombosis.

In another embodiment, for clinical islet transplantation to mature into a safe and practical standard-of-care, alternative sites for implantation, capable of supporting the long-term endocrine function of isolated islets are needed. In another embodiment, the subcutaneous space is an attractive islet transplant site. In another embodiment, the present invention provides a novel technique for implantation of the islet allograft in the subcutaneous space, as a safe and practical site for islet engraftment. In another embodiment, islets transplanted subcutaneously within a collagen matrix were capable for rendering diabetic recipients euglycemic with normal glucose tolerance tests following transplantation.

### Subcutaneous

In one embodiment, the present invention provides a method for maintaining the viability of transplanted islet cells in-vivo, comprising transplanting a composition of the present invention, subcutaneously into a subject (Example 2). In one embodiment, the present invention provides a method for increasing insulin production in a subject, comprising transplanting a composition of the present invention, subcutaneously into a subject. In one embodiment, insulin production is measured by the methods illustrated in Example 7. In one embodiment, the present invention provides a method of treating diabetes in a subject, comprising transplanting a composition of the present invention, subcutaneously into a subject, thereby treating diabetes.

In one embodiment, the methods of the present invention comprise transplanting subcutaneously to a dorsal area in a subject. In one embodiment, the methods of the present invention comprise transplanting subcutaneously to a ventral area in a subject. In one embodiment, the methods of the present invention comprise transplanting subcutaneously to a dorsal-lateral area in a subject. In one embodiment, the methods of the present invention comprise transplanting subcutaneously to a ventral-lateral area in a subject. In one embodiment, the methods of the present invention comprise transplanting subcutaneously in the abdomen in a subject (Example 2).

In one embodiment, the methods of present invention provide that the composition of the present invention is delivered by a subcutaneous injection. In one embodiment, the methods of present invention provide that a needle is inserted just under the skin. In one embodiment, the methods of present invention provide that the composition can then be delivered into the subcutaneous tissue.

In one embodiment, the methods of present invention provide that the composition of the present invention is delivered through a pedicle flap. In one embodiment, the methods of present invention provide that the pedicle is denuded of epithelium and buried in the subcutaneous tissue in the recipient area.

### Retroperitoneal Space

In one embodiment, the present invention provides a method for maintaining the viability of transplanted islet cells in-vivo, comprising the step of transplanting a composition comprising islet cells into the retroperitoneal space of a subject. In one embodiment, the present invention provides a method for increasing insulin production in a subject, comprising transplanting a composition comprising islet cells, into the retroperitoneal space of a subject. In one embodiment, the present invention provides a method of treating diabetes in a subject, comprising transplanting a composition comprising islet cells, into the retroperitoneal space of a subject, thereby treating diabetes. In one embodiment, the retroperitoneal space is the space between the posterior parietal peritoneum and the posterior abdominal wall, containing the kidneys, adrenal glands, ureters, duodenum, ascending colon, descending colon, pancreas and the large vessels and nerves.

In one embodiment, the present invention provides a method for maintaining the viability of transplanted islet cells in-vivo, comprising the step of transplanting a composition comprising islet cells and an extracellular matrix component into the retroperitoneal space of a subject. In one embodiment, the present invention provides a method for increasing insulin production in a subject, comprising transplanting a composition comprising islet cells and an extracellular matrix component, into the retroperitoneal space of a subject. In one embodiment, the present invention provides a method of treating diabetes in a subject, comprising transplanting a composition comprising islet cells and an extracellular matrix component, into the retroperitoneal space of a subject, thereby treating diabetes. In one embodiment, the retroperitoneal space is the space between the posterior parietal peritoneum and the posterior abdominal wall, containing the kidneys, adrenal glands, ureter, duodenum, ascending colon, descending colon, pancreas and the large vessels and nerves.

In one embodiment, the methods of present invention provide that the composition of the present invention is administered retroperitoneally. In one embodiment, the methods of present invention provide that the composition of the present invention is administered retroperitoneally in proximity to the kidneys (Example 3). In one embodiment, the methods of present invention provide that the composition of the present invention is administered retroperitoneally in proximity to the adrenal glands. In one embodiment, the methods of present invention provide that the composition of the present invention is administered retroperitoneally in proximity to the ureter. In one embodiment, the methods of present invention provide that the composition of the present invention is administered retroperitoneally in proximity to the duodenum. In one embodiment, the methods of present invention provide that the composition of the present invention is administered retroperitoneally in proximity to the ascending colon. In one embodiment, the methods of present invention provide that the composition of the present invention is administered retroperitoneally in proximity to the descending colon. In one embodiment, the methods of present invention provide that the composition of the present invention is administered retroperitoneally in proximity to the pancreas.

In one embodiment, the methods of present invention provide that the composition of the present invention is delivered by a retroperitoneal injection. In one embodiment, the methods of present invention provide that a needle is inserted through subcutaneous layers, muscle layers, lumbodorsal fascia and finally entering the retroperitoneal space. In one embodiment, the methods of present invention provide that the composition can then be delivered into the retroperitoneal space.

In one embodiment, the methods of present invention provide that the composition of the present invention is delivered through a muscle-split incision (Example 3). In one embodiment, the methods of present invention provide that the 0.2-5 cm flank muscle-split incision is made, and the wound is deepened into the retroperitoneal space. In one embodiment, the methods of present invention provide that the composition of the present invention is administered through the muscle-split incision to the retroperitoneal space.

In one embodiment, the present invention comprises a micro-organ. In one embodiment, the preparation of a micro-organ is illustrated in Example 8. In one embodiment, a micro-organ comprises islet cells of the invention. In one embodiment, a micro-organ comprises pancreatic β cells of the invention. In one embodiment, a micro-organ comprises pancreatic β cells of the invention and a nutrient medium of the invention. In one embodiment, a micro-organ comprises a composition of the present invention. In one embodiment, a micro-organ comprises a composition of the present invention comprising islet cells, a nutrient medium, and an ECM component. In one embodiment, a micro-organ comprises a composition of the present invention comprising islet cells, a nutrient medium, and collagen.

In one embodiment, a micro-organ of the present invention is transplanted to a subject in need thereof. In one embodiment, a micro-organ of the present invention is transplanted intravenously, intraperitonealy, retroperitoneally, intramuscularly, or subcutaneously. In some embodiments, the micro-organ is in a liquid form. In some embodiments, the micro-organ is in a solid form.

### EXAMPLES

### Example 1

### Transplanting to Diabetic Mice

Several groups of diabetic recipients were transplanted: 1) diabetic B6/scid recipients of B6 wild-type islets, 2) diabetic wild-type B6 recipients of B6 wild-type islets, 3) diabetic wild-type B6 recipients of allogeneic BALB/c islet under cover of anti-lymphocyte serum (ALS) to prevent rejection. The results of these studies demonstrated indefinite survival of the islet isografts in groups 1 and 2, indicating the efficacy of our islet matrix in supporting islet endocrine function in the subcutaneous compartment. Group 3 recipients were rendered euglycemic with normal glucose tolerance tests within 24 hrs. Following transplantation and enjoyed prolonged survival for 20-30 days under cover of ALS. This result confirmed the capacity of the subcutaneous space for supporting islet allograft function and demonstrated that islets embedded in the collagen matrix are not immunologically privileged; similar to islet allografts transplanted in various other anatomic site. Table 1 shows the islet survival period in Subcutaneous isolated islet transplantation using a collagen matrix. Isolated islets were mixed with a 250 mcl solution of collagen-I and injected into the subcutaneous plane in the anterior abdominal wall of STZ-induced diabetic mice. In no instance did isolated islets without the collagen solution survive to promote a cure. Allogeneic islets enjoyed long-term survival under cover of ALS therapy, indicating that the subcutaneous space is amenable immunomodulation.

TABLE 1

| | | | | | Islet |
|---|---|---|---|---|---|
| | | | | | Survival |
| Group | Donor | Recipient | Islet Prep | Treatment | (days) |
| 1 | B6 | B6/scid | Islet + Collagen | None | >120 |
| | | | | | (n = 13) |
| 2 | B6 | B6 WT | Islet + Collagen | None | >250 |
| | | | | | (n = 40) |
| 3 | BALB/c | B6 WT | Islet + Collagen | ALS × 2 | 23, 42, |
| | | | | doses | >147 × 2 |
| 4 | B6 | B6/scid | Islet Alone | None | No cure |
| | | | | | (n = 12) |
| 5 | B6 | B6 | Islet Alone | None | No cure |
| | | | | | (n = 16) |

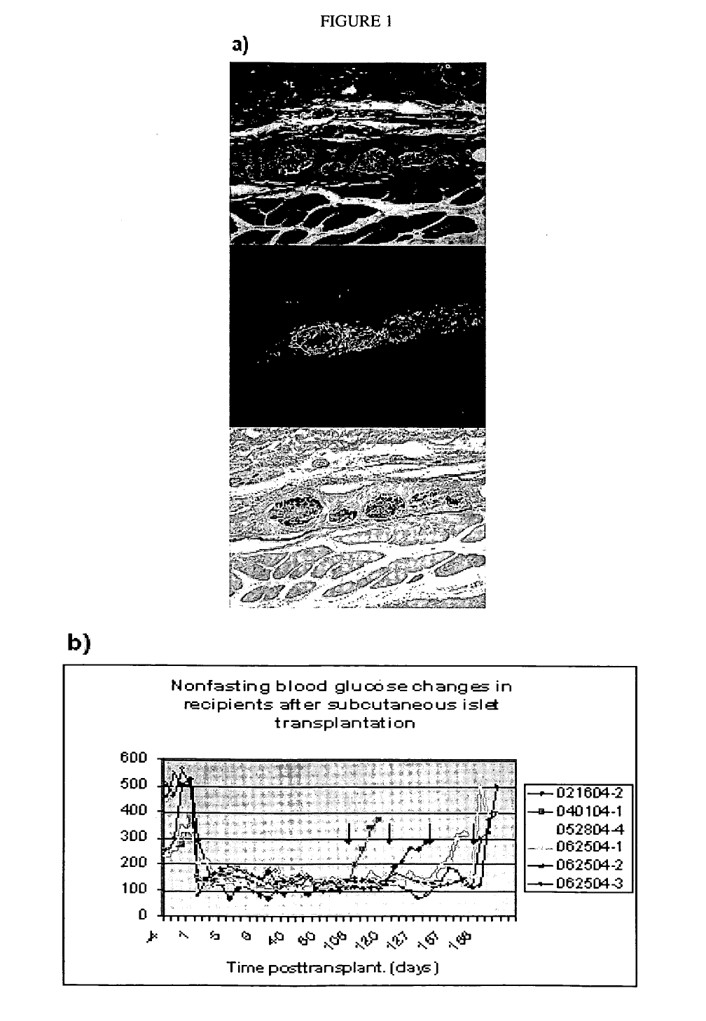

The survival of islet isografts within a collagen matrix following subcutaneous transplantation is further demonstrated in FIG. 1. This analysis demonstrates the persistence of healthy subcutaneous islets upon transplantation in our collagen-I matrix (FIG. 1A). Random a.m. blood glucose levels in a cohort of B6 mice transplanted subcutaneously with syngeneic isolated islet is shown in FIG. 1B. The arrows indicate the timing of islet-bearing anterior abdominal wall excision, which in all cases precipitated diabetes recurrence. Based on these results, isolated human islets in collagen matrix were transplanted into diabetic B6/scid recipients.

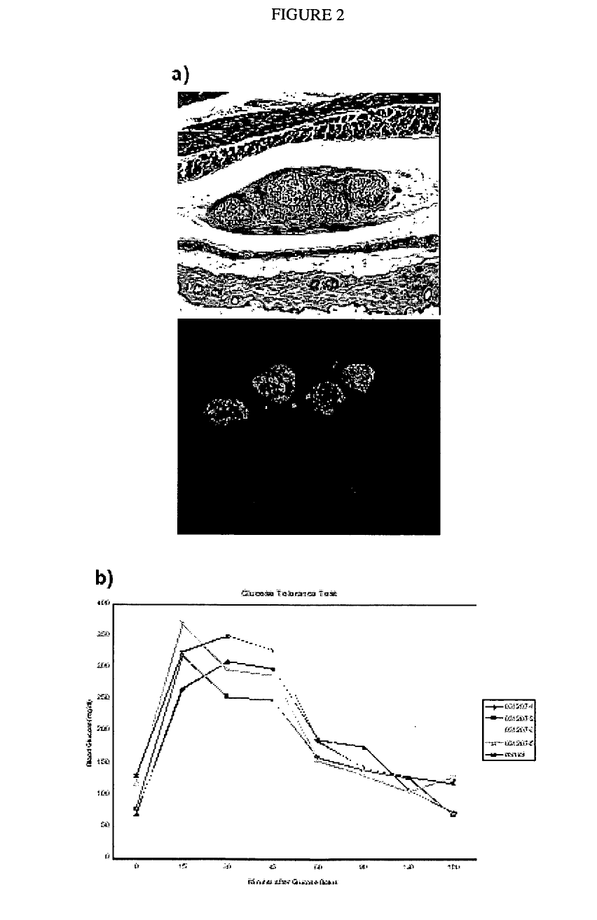

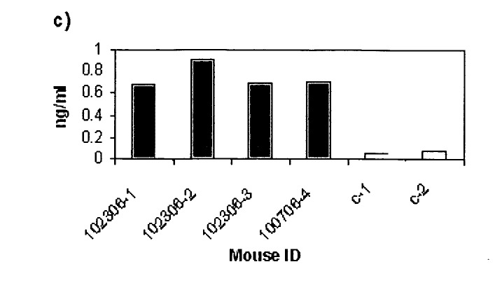

These human islets enjoyed permanent survival in the subcutaneous compartment and rendered the diabetic mice indefinitely euglycemic (>200 days) with normal glucose tolerance test (FIG. 2).

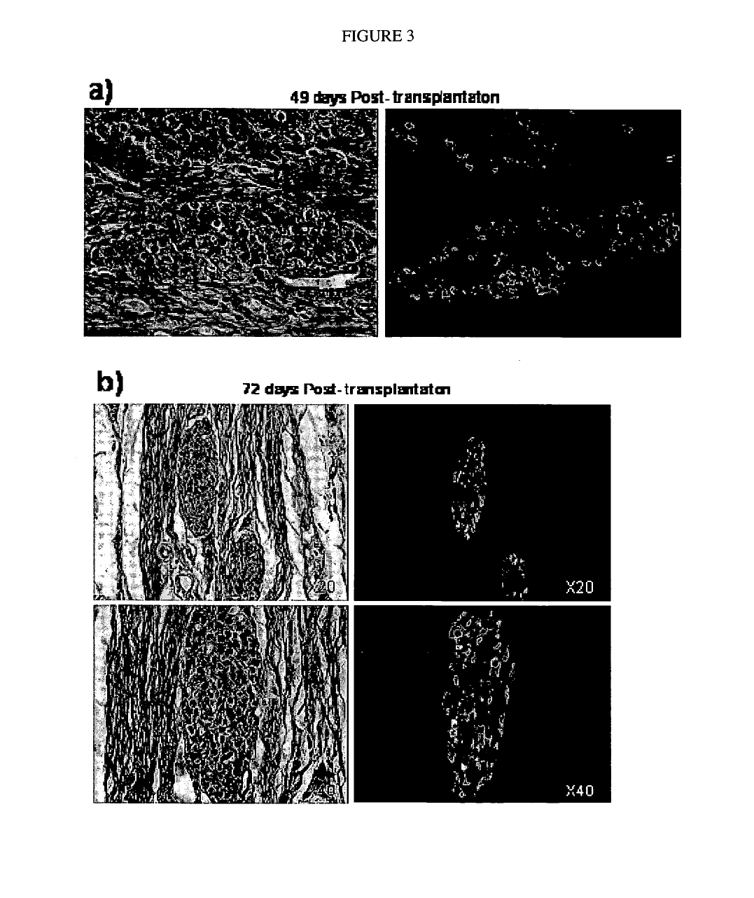

Results of subcutaneously transplanted human islet in STZ-induced diabetic B6/scid recipients are shown in FIG. 2. Excision of the islet bearing anterior abdominal wall was performed at >200 days following transplantation and led to prompt recurrence of diabetes in the recipient. These results provided the rationale to translate this technique into a non-human primate model of islet transplantation. Therefore, in a preliminary experiment autologous islets contained in collagen matrix were subcutaneously transplanted into two recipients in whom a subtotal pancreatectomy was performed. This preliminary experiment provided us with the opportunity to assess the long-term viability of isolated non-human primate islets, embedded in the collagen matrix, within the subcutaneous space. Therefore, the recipients underwent skin biopsies at the implantation site 49 and 250 days following transplantation (FIG. 3). This experiment demonstrated abundant viable insulin and glucagon producing islets, free of inflammation and fibrosis, in the subcutaneous space.

The biopsy of abdominal wall of cynomolgus monkeys after autologous islet transplantation into the subcutaneous space is shown in FIG. 3. This result again, indicates that the method as described herein promotes successful subcutaneous islet transplantation.

In conclusion, this example demonstrates a method that consistently promotes successful subcutaneous islet transplantation. This strategy is now translatable to human islet transplantation for treatment of type 1 diabetes mellitus.

### Example 2

### Pancreatic Islet Cells Transplantation in the Abdominal Subcutaneous Space

Rat primary pancreatic islet cells were obtained as described in Example 4. Collagen gel was prepared according to the descriptions of Example 5. The pancreatic islet cells were grown in monolayers for 48 h. Then 0.5 ml of DMEM containing 10⁶ islet pancreatic cells was combined with collagen gel. Diabetic rats fed on normal diet were divided into two groups. Group 1 received a composition comprising a mixture of islet cells in 0.5 ml DMEM and collagen. Group 2 received a composition, comprising only 10⁶ islet pancreatic cells in 0.5 ml DMEM. Both groups received the composition subcutaneously to the abdomen through a needle inserted just under the skin. Samples of histological sections were obtained and primary islets cells were isolated from the transplants of Groups 1 and 2, at 2 days, 4 days, 7 days, 10 days, 14 days, 21 days, and 30 days post transplantation. All histological samples were stained with hematoxylin-eosin. A month after being transplanted the transplants containing collagen retained over 85% viability of the transplanted pancreatic islet cells wherein transplants containing islet pancreatic cells in 0.5 ml DMEM only retained less then 5% viability. The post-transplanted primary islets cells were further assessed for their functionality using radioimmunoassay as described in Example 7. 14 days post transplantation the transplants containing collagen secreted more than a 1000 fold more insulin than the transplants containing islet pancreatic cells in 0.5 ml DMEM only (normalized to the number of viable cells). A constant daily increase from 2 days up to 30 days, in insulin production of about 7% was recorded for the transplants containing collagen. Further, blood glucose was monitored daily.

### Example 3

### Pancreatic Islet Cells Transplantation in the Retroperitoneal Space

Rat primary pancreatic islet cells were obtained as described in Example 4. Collagen gel was prepared according to the descriptions of Example 5. The pancreatic islet cells were grown in monolayers for 48 h. Then 0.5 ml of DMEM containing 10⁶ islet pancreatic cells was combined with collagen gel. Diabetic rats fed on normal diet were divided into two groups. Group 1 received a composition comprising a mixture of islet cells in 0.5 ml DMEM and collagen. Group 2 received a composition, comprising only 10⁶ islet pancreatic cells in 0.5 ml DMEM. Both groups received the composition retroperitoneally in close proximity to the kidneys. The composition was delivered by a pump through a 0.5 cm muscle-split incision deepened into the retroperitoneal space. Samples of histological sections were obtained and primary islets cells were isolated from the transplants of Groups 1 and 2, at 2 days, 4 days, 7 days, 10 days, 14 days, 21 days, and 30 days post transplantation. All histological samples were stained with hematoxylin-eosin. A month after being transplanted the transplants containing collagen retained over 82% viability of the transplanted pancreatic islet cells wherein transplants containing islet pancreatic cells in 0.5 ml DMEM only retained less then 2% viability. The post-transplanted primary islets cells were further assessed for their functionality using radioimmunoassay as described in Example 7. 14 days post transplantation the transplants containing collagen secreted more than a 1000 fold more insulin than the transplants containing islet cells in 0.5 ml DMEM only (normalized to the number of viable cells). A constant daily increase from 2 days up to 30 days, in insulin production of about 5.3% was recorded for the transplants containing collagen. Further, blood glucose was monitored daily. Group 1 had normal blood glucose levels from 4 days after transplantation until termination of the experiment, wherein Group 2 blood glucose levels fluctuated throughout the experimental period.

### Example 4

### Isolation of Pancreatic Islet Cells

Primary pancreatic islet cells are obtained from a pancreas digested with collagenase in Ca²⁺-containing Hanks buffer wherein islets of Langerhans are separated from exocrine tissue by discontinuous density-gradient centrifugation (Histopaque 1077 from Sigma). Islets are treated with trypsin, and cells are sorted from non-β cells by size and FAD auto-fluorescence using a fluorescence-activated cell sorter (FACS). This method is shown by classical double-immunofluorescence techniques to yield one population consisting of 95% β cells, and a second population with 93% non-β cells.

### Example 5

### Preparation of a Collagen Gel

Type I collagen is solubilized by stirring adult rat tail tendons for 48 h at 4° C. (˜in a sterile 1:1,000 (vol./vol.) acetic acid solution (300 ml for 1 g of collagen)). The resulting solution is filtered through sterile triple gauze and centrifuged at 16,000 g for 1 h at 4° C. The supernatant is then extensively dialyzed against 1/10 Eagle's minimal essential medium (Gibco, Grand Island, N.Y.) and stored at 4° C. Gels of reconstituted collagen fibers are prepared by simultaneously raising the pH and ionic strength of the collagen solution. This is achieved by quickly mixing 7 volumes of cold collagen solution with 1 volume of 10× Eagle's minimal essential medium and 2 volumes of sodium bicarbonate (11.76 mg/ml) in a sterile flask kept on ice to prevent immediate gelation. The cold mixture is then dispensed into 35-mm plastic culture dishes (Falcon Plastics, Div. of Bioquest, Oxnard, Calif.) (˜0.8 ml per dish) and allowed to gel for 10 min at 37° C.

### Example 6

### Preparation of a Composition Comprising Islet Cells and Collagen

Islet cells are isolated by enzymatic dissociation as described in Example 4 and plated onto 100 mm plastic dishes in DMEM supplemented with 10% heat-inactivated fetal calf serum, 400 U/ml sodium penicillin, and 16.7 mM glucose. After 16 h of incubation at 37° C. the culture medium is removed and replated in new dishes. Following a second “sedimentation” period of 6 h, the medium was transferred to 35 mm collagen-coated dishes, and the cells were allowed to attach and spread on the surface of the gels for 24 h at 37° C. After removing the culture medium and unattached cells, ˜0.8 ml of the cold collagen mixture described in Example 5 poured on the top of the first gel and allowed to polymerize for 10 min at 37° C. Fresh medium is added after the collagen had gelled, and is renewed at 48 h intervals.

### Example 7

### Assessment of Transplants Functionality

Islets cells transplanted subcutaneously in a collagen matrix are removed 2 days, 3 days, 4 days, 6 days, 7 days, 10 days, 14 days, 21 days, and 30 days post transplantation. To release the reorganized clusters of endocrine cells from the surrounding collagen matrix, cultures are incubated for 1 h at 37° C. with 0.1% collagenase in culture medium. Triplicate groups of 5 islets each are placed in single well of 24 well plate in KRBH. The plates are incubated at 37° C. in a CO₂ incubator for 1 h with 5×5 mM and 16 mM glucose respectively. The supernatant is collected and stored at −20° C. and assayed for basal insulin level. Radioimmunoassay is carried out using Radioimmunoassay kit (Diagnostic Products Corporation, Los Angles, USA) and insulin content of all the stored samples are determined.

### Example 8

### Preparation of Micro-Organ Cultures From the Pancreas

Pig pancreas is removed and then cut into sections of 300 μm in thickness, 4 mm in width and 2 mm in depth. The micro-explants are grown in culture for several time periods from 2 to 18 days. Seven micro-organs are placed in each of 96 wells of a plate in 150 μl DMEM under 5% CO₂ at 37° C.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising pancreatic islet cells and an extracellular matrix component.

2. The composition of claim 1, wherein said islet cells are substantially β cells.

3. The composition of claim 1, further comprising a cell culture medium.

4. The composition of claim 1, wherein said extracellular matrix component is collagen, elastin, fibrillin, fibronectin, laminin, proteoglycans, or a mixture thereof.

5. The composition of claim 4, wherein said collagen is collagen type IV, collagen type I or their combination.

6. The composition of claim 1, further comprising entactin, polylysinearginine, polylysine, proline, nicotinamide, transferrin, insulin, insulin-like growth factors, glucocorticoid steroid, L-glutamine, D-galactose, D-glucose, or a mixture thereof.

7. The composition of claim 1, wherein said pancreatic islet cells are primary pancreatic islet cells, pancreatic islet cell lines, genetically-transformed pancreatic islet cells, pancreatic islet cells obtained from neoplastic sources, fetal pancreatic islet cells, or primary pancreatic islet carcinoma cells.

8. The composition of claim 1, wherein said composition is a transplantable composition.

9. A method for maintaining the viability of transplanted pancreatic islet cells in-vivo, comprising the step of: transplanting a composition comprising pancreatic islet cells and an extracellular matrix component into a subject.

10. The method of claim 9, wherein , wherein said transplanted pancreatic islet cells are primary pancreatic islet cells, pancreatic islet cell lines, genetically-transformed pancreatic islet cells, pancreatic islet cells obtained from neoplastic sources, fetal pancreatic islet cells, primary pancreatic islet carcinoma cells or their combination.

11. The method of claim 9, wherein said transplanted pancreatic islet cells are substantially β cells.

12. The method of claim 9, wherein said transplanted pancreatic islet cells express increased levels of insulin as compared to dedifferentiated cells.

13. The method of claim 9, wherein said transplanted pancreatic islet cells have the ability to secrete insulin in response to glucose.

14. The method of claim 9, wherein said extracellular matrix component is collagen, elastin, fibrillin, fibronectin, laminin, proteoglycans, or a mixture thereof.

15. The method of claim 14, wherein said collagen is collagen type IV, collagen type I or their combination.

16. The method of claim 8, wherein the step of transplanting is via subcutaneous transplantation.

17. The method of claim 14, wherein the subcutaneous transplantation is in the abdomen.

18. The method of claim 8, wherein the step of transplanting is in the retroperitoneal space.

19. A method for increasing insulin production in a subject, said method comprising: transplanting the composition of claim 1.

20-30. (canceled)

31. A method of treating diabetes in a subject, said method comprising: transplanting the composition of claim 1.

32. The method of claim 31, wherein said composition further comprises a cell culture medium.

33. The method of claim 31, wherein said composition further comprises fibronectin, laminin, entactin, polylysinearginine, polylysine, proline, nicotinamide, transferring, insulin, insulin-like growth factors, glucocorticoid steroid, L-glutamine, D-galactose, D-glucose, or a mixture thereof.

34. The method of claim 31, wherein said transplanting is subcutaneously.

35. The method of claim 31, wherein said transplanting subcutaneously is subcutaneously in the abdomen.

36. The method of claim 31, wherein said transplanting is in the retroperitoneal space.

37. The method of claim 31, wherein said pancreatic islet cells are substantially β cells.

38-64. (canceled)
